(19) United States
(12) Patent Application Publication
(10) Pub. No.: US 2006/0229475 A1
(43) Pub. Date: Oct. 12, 2006

(54) Synthesis of hydroxylated hydrocarbons

(76) Inventors: Michael J. Weiss, Santa Barbara, CA (US); Shouli Sun, Santa Barbara, CA (US)

Correspondence Address: JOHN D. CARPENTER;CHRISTIE, PARKER & HALE L.L.P., 350 W. COLORADO BOULEVARD, SUITE 500, PASADENA, CA, 91105, US

(21) Appl. No.: 11/103,326

(22) Filed: Apr. 11, 2005

(51) Int. Cl. C07C 29/124

(52) U.S. Cl. 568/891

(57) ABSTRACT

Ethylene glycol, other diols, triols, and polyols are made in an efficient manner by reacting dibromides with water in the presence of a metal oxide. An integrated process of dibromide formation, alcohol synthesis, metal oxide regeneration, and bromine recycling is also provided.

### FIELD OF THE INVENTION

The invention relates generally to methods of making hydrocarbons substituted with two or more hydroxyl groups, including aliphatic and aromatic diols, triols, and polyols, and in particular relates to the synthesis of such compounds from the reaction of dibromides and water in the presence of a metal oxide. An integrated process using hydrocarbon feedstocks and metal oxide and bromine regeneration is also disclosed.

### BACKGROUND OF THE INVENTION

Glycols and related products are important industrial chemicals. Ethylene glycol, for example, is one of the largest volume commodity organic chemicals and is used in antifreeze mixtures and in the production of polyester. The triol glycerin (glycerol) is used throughout the chemical industry, for example, in the manufacture of alkyd resins, pharmaceuticals and cosmetics, foodstuffs, and adhesives and glues. More highly hydroxylated polyols (polyhydric alcohols) are used to make urethane foams, coatings, and as humectants and plasticizers.

Glycols can be made by treating an alkene with an oxidizing agent, such as peroxyformic acid. Similarly, a vicinal glycol can be made by treating an alkene with a cold, alkaline solution of potassium permanganate (KMnO₄). When KMnO₄ is used, manganese oxide precipitates out of solution as a product.

Currently, ethylene glycol is made on an industrial scale through the partial oxidation of ethylene to ethylene oxide (EO) and subsequent hydration. The process has several disadvantages, including low conversions in the partial oxidation step, due to the need to avoid complete combustion to carbon dioxide; the need for a costly separation of the approximately 10-fold excess of water used in the hydration of EO (often accomplished by vacuum distillation); and the formation of significant amounts of di (˜9%) and tri (˜1%) glycols.

Synthetic processes are also used to make other diols, triols, and polyols. For example, although glycerin is often produced as a by-product of the manufacture of soap, or from the hydrolysis of fats and oils, a significant amount is also made by various synthetic routes, starting with propylene. Other polyols are obtained from natural sources, or made by the catalytic hydrogenation of sugars, or by reacting formaldehyde with other aldehydes.

Given the importance of diols, triols, and more highly hydroxylated materials, a new, more universal synthetic route to their production would be a welcome development. Particularly useful would be a process that uses lower cost starting materials (e.g., alkanes and other hydrocarbons), avoids the inefficient partial oxidation of olefins, and the inefficient hydration of olefin oxides, avoids precipitate formation, and requires fewer (and less expensive) purification steps.

### SUMMARY OF THE INVENTION

The present invention provides methods of making hydrocarbons substituted with two or more hydroxyl groups, including aliphatic and aromatic diols, triols, and polyols. According to one aspect of the invention, a hydroxylated hydrocarbon having two or more hydroxyl groups (sometimes referred to as a “hydroxylate”) is made by allowing a brominated hydrocarbon having at least two bromine atoms to react with water or steam in the presence of a metal oxide to form a hydroxylated hydrocarbon having two or more hydroxyl groups. For example, ethylene glycol can be made by reacting 1,2-dibromoethane with water in the presence of a metal oxide, e.g., copper oxide. Preferably, the metal oxide is used in solid form.

In a second aspect of the invention, a hydroxylated hydrocarbon having two or more hydroxyl groups is made by forming a brominated hydrocarbon (e.g., by allowing a hydrocarbon feedstock to react with bromine), and then allowing the brominated hydrocarbon to react with water or steam in the presence of a metal oxide to form a hydroxylated hydrocarbon having two or more hydroxyl groups. The invention also provides an “integrated process” in which the metal oxide and bromine are regenerated. For example, in one embodiment of the invention, ethane is brominated with molecular bromine; the resulting 1,2-dibromoethane is allowed to react with water in the presence of a metal oxide, resulting in metal bromide(s) and ethylene glycol; and the metal oxide and bromine are regenerated by allowing metal bromide(s) to react with air or oxygen.

### BRIEF DESCRIPTION OF THE DRAWING

These and other features and advantages of the invention will become better understood when considered in conjunction with the following detailed description, and by making reference to the appended drawing, which is a schematic illustration of an integrated process for making hydroxylated compounds according to one embodiment of the invention.

### DETAILED DESCRIPTION OF THE INVENTION

According to a first aspect of the invention, a brominated hydrocarbon (sometimes called a “bromocarbon”) having two or more bromine atoms is allowed to react with water or steam in the presence of a metal oxide to form a hydroxylated hydrocarbon having at least two hydroxyl groups (i.e., a di-, tri-, or polyhydric alcohol, referred to, respectively, as a diol, triol, or polyol). Small amounts of other species (e.g., monohydric alcohols, bromine-substituted alcohols, alkanes, aldehydes, etc.) may also be produced. Preferably, the reaction is carried out in either the gas or liquid phase.

Several classes of brominated hydrocarbons having two or more bromine atoms per molecule are suitable for use in the present invention, including aliphatic bromocarbons (which may be saturated or unsaturated), aromatic bromocarbons, and mixed aliphatic-aromatic compounds. The list includes alkyl dibromides (dibromoalkanes), alkyl tribromides (tribromoalkanes), and more highly brominated alkanes (polybrominated alkanes); di-, tri-, and polybrominated alkenes and alkynes; aromatic dibromides (dibromoarenes), aromatic tribromides (tribromoarenes), and polybrominated aromatic hydrocarbons; and mixed aliphatic-aromatic compounds having two or more bromine atoms. Also included are vicinal dibromides, non-vicinal dibromides, and perbrominated hydrocarbons.

Vicinal dibromides of particular interest are those having the generic formula BrCH₂CH(Br)—R, where R is hydrogen, C₁₋₁₆ alkyl (more preferably C₁₋₄ alkyl), or phenyl; or R—CH(Br)—CH(Br)—R′, where R and R′ are independently selected from the group consisting of hydrogen, C₁₋₁₆ alkyl (more preferably C₁₋₄ alkyl), and phenyl. The term “alkyl” is used in its broadest sense and includes normal, branched, and cyclic species.

A non-limiting list of specific examples of brominated hydrocarbons having two or more bromine atoms per molecule includes 1,2-dibromoethane (ethylene dibromide), 1,2-dibromopropane (vic-propylene bromide), 1,3-dibromopropane, 2,3-dibromobutane, 1,2-dibromocyclohexane, 1,4-dibromobutane, 1,2,3-tribromopropane, 1,4-dibromobut-2-ene, 2,3,4,5-tetrabromohexane, brominated polyolefins (e.g., the reaction product of bromine and a polyolefin having an average molecular weight prior to bromination of 250-5000, preferably 500-2500), o-, m-, and p-dibromobenzene, and 1,2-dibromoethenylbenzene.

The reaction of a brominated hydrocarbon and water is carried out in the presence of at least one metal oxide (preferably in solid form) that facilitates the production of one or more hydroxylates. A nonlimiting list of metal oxides includes oxides of copper, magnesium, yttrium, nickel, cobalt, iron, calcium, vanadium, molybdenum, chromium, manganese, zinc, lanthanum, tungsten, tin, indium, and bismuth. Also included are doped metal oxides. For example, in one embodiment of the invention, any of the above-listed metal oxides is doped with an alkali metal or an alkali metal halide, preferably to contain 5-20 mol % alkali.

Particularly preferred are (i) binary oxides such as CuO, MgO, Y₂O₃, NiO, Co₂O₃, and Fe₂O₃; (ii) alkali metal-doped mixed oxides, e.g., oxides of copper, magnesium, yttrium, nickel, cobalt, or iron, doped with one or more alkali metals (e.g., Li, Na, K, Rb, Cs) (most preferably with 5-20 mol % alkali content); (iii) alkali metal bromide-doped oxides, most preferably oxides of copper, magnesium, yttrium, nickel, cobalt, or iron (alkali metal bromide dopants include LiBr, NaBr, KBr, RbBr, and CsBr); and (iv) supported versions of any of the aforementioned oxides and doped oxides. Nonlimiting examples of suitable support materials include zirconia, titania, alumina, and silica. One or more metal oxides (with or without alkali dopants) are used.

The metal oxide is perhaps best characterized as a “cataloreactant,” rather than a true catalyst, as it is converted to a metal bromide during the reaction. (For a generic metal oxide, “MOₓ,” the metal bromide(s) expected to be formed has a formula “MBr₂ₓ.”) However, treating the metal bromide with oxygen or air (preferably at an elevated temperature) regenerates the metal oxide. The reaction may be generalized as MBr₂ₓ+O₂→MOₓ+Br₂, where the value of x depends on the oxidation state of the metal.

Table 1 identifies the metal bromides that are believed or predicted to be formed as a result of the metal oxide-facilitated reaction of a brominated hydrocarbon and water.

TABLE 1
Predicted Metal Bromides Generated from a Brominated
Hydrocarbon and Selected Metal Oxides and Dopants

| | Metal Bromide |
|---|---|
| Metal Oxide | |
| CuO | CuBr, CuBr₂ |
| MgO | MgBr₂ |
| Y₂O₃ | YBr₃ |
| NiO | NiBr₂ |
| Co₂O₃ | CoBr₂ |
| Fe₂O₃ | FeBr₂, FeBr₃ |
| CaO | CaBr₂ |
| VO | VBr₂, VBr₃ |
| MoO₂ | MoBr₂, MoBr₃, MoBr₄ |
| Cr₂O₃ | CrBr₂, CrBr₃ |
| MnO | MnBr₂ |
| ZnO | ZnBr₂ |
| La₂O₃ | LaBr₃ |
| WO₂ | WBr₂, WBr₅, WBr₆ |
| SnO | SnBr₂, SnBr₄ |
| In₂O₃ | InBr₃ |
| Bi₂O₃ | BiBr₃, BiOBr |
| Alkali Metal Dopant | |
| Li | LiBr |
| Na | NaBr |
| K | KBr |
| Rb | RbBr |
| Cs | CsBr |

Without being bound by theory, it is believed that the alkali metal in an alkali metal-doped oxide of copper, magnesium, yttrium, nickel, cobalt, or iron (and possibly others) will, upon interaction with a bromocarbon, be converted into an alkali metal bromide (LiBr, NaBr, KBr, etc.) and remain as such. It is further believed that such dopants will not provide a sink for bromine, though they will likely influence the chemistry of the metal oxide. Metal oxide supports, such as zirconia, titania, alumina, silica, etc., are not expected to be converted to their respective bromides.

Advantageously, the metal oxide-facilitated synthesis of a hydroxylate uses a stoichiometric amount of water (or steam). Hence, costly separation of product(s) from water is avoided.

Another advantage of the invention is the high selectivity of product formation, as evidenced by the conversion of an aliphatic dibromide to a diol. In the ideal case, every bromine atom is replaced by a hydroxyl group, and no rearrangement occurs. Thus a vicinal dibromide having the formula BrCH₂CH(Br)—R or R—CH(Br)—CH(Br)—R′, where R and R′ are, independently, hydrogen, C₁₋₁₆ alkyl, or phenyl, is converted to a vicinal diol having the formula HOCH₂CH(OH)—R or R—CH(OH)—CH(OH)—R′, respectively; a non-vicinal dibromide is converted a non-vicinal diol; o-, m-, and p-dibromobenzene are converted, respectively, to catechol, resorcinol, and hydroquinone; a polybrominated polyolefin is converted to a polyhydroxylated polyolefin; and so forth. The invention includes the production of all such hydroxylates, as well as hydroxylates where rearrangement has occurred

Selectivities above 80% have been observed for a number of hydroxylate syntheses, for example, the conversion of 1,2-dibromoethane to ethylene glycol, and 1,3-dibromoethane to 1,3-propane diol (see Examples 1 and 3 below). More generally, the invention provides selectivities greater than or equal to 50%, preferably greater than 60%, more preferably greater than 70%, and even more preferably greater than 80%. For a hydroxylate synthesis utilizing an aliphatic dibromide starting material, the following product distributions have been observed, or are predicted:

I. A major amount (60-99%) of a desired diol;

II. A minor amount (1-20%) of byproducts, including
(a) epoxides (for vicinal diols), (i.e. ethylene oxide in an ethylene glycol process)
(b) aldehydes (e.g. acetaldehyde from 1,2-dibromoethane in an ethylene glycol process)
(c) ketones (e.g. acetone in a propane diol process
(d) cyclic ethers (e.g. oxetane in a 1,3-propane diol process; also 1,4-dioxane in an ethylene glycol process)
(e) diglycols (i.e., diethylene glycol in a mono-ethylene glycol process)
(f) bromohydrins (i.e., CH₂BrCH₂OH in an ethylene glycol process)
(g) olefins (i.e., ethylene in an ethylene glycol process)
(h) brominated olefins (i.e., vinyl bromide in an ethylene glycol process)
(i) carbon dioxide
(j) organic acids (i.e., acetic acid in an ethylene glycol process)
(k) tri- and higher glycols.

The most significant byproducts are predicted to be carbon dioxide, epoxides, aldehydes, bromohydrins, and digylcols.

For vicinal diols (glycols), the selectivity of the reaction should favor monoglycols over diglycols over triglycols; with expected product ratios of 90:9:1, (mono:di:tri).

Without being bound by theory, one can identify the following likely mechanisms for diol formation from aliphatic dibromides:

I. Epoxide Route
(i) A vicinal dibromide reacts with metal oxide to form epoxide and metal bromide: RBr₂+MO→RO (epoxide)+MBr₂
(ii) The epoxide is hydrated in situ to glycol: RO+H₂O→R(OH)₂

II. Bromohydrin Route
(i) A dibromide reacts with metal oxide and water, substituting a hydroxyl for a bromide, forming a bromohydrin and metal bromide/hydroxide:
   RBr₂+H₂O+MO→RBrOH+M(OH)Br
   Alternate: RBr₂+MOH→RBrOH+MBr
   Alternate: RBr₂+MOHBr→RBrOH+MBr₂
(ii) The bromohydrin reacts with metal oxide and water or metal hydroxide to make glycol:
   RBr(OH)+H₂O+MO→R(OH)₂+MBrOH
   RBr(OH)+MBrOH→R(OH)₂+MBr₂
   RBr(OH)+MOH→R(OH)₂+MBr

In a second aspect of the invention, a hydroxylated hydrocarbon having two or more hydroxyl groups is produced in two steps: First, a hydrocarbon is brominated to generate a brominated hydrocarbon having at least two bromine atoms. Second, the brominated hydrocarbon is reacted with water in the presence of a metal oxide to form a diol, triol, or polyol, and one or more metal bromides. One or more additional steps may also be employed. Nonlimiting examples include the separation of any undesired isomers produced in the bromination step (optionally followed by isomerization/rearrangement to yield the desired isomer, which can then be returned to the reactor and allowed to react with water to form additional product); separation of the metal bromide from the alcohol(s); and regeneration of the metal oxide and bromine using air or oxygen.

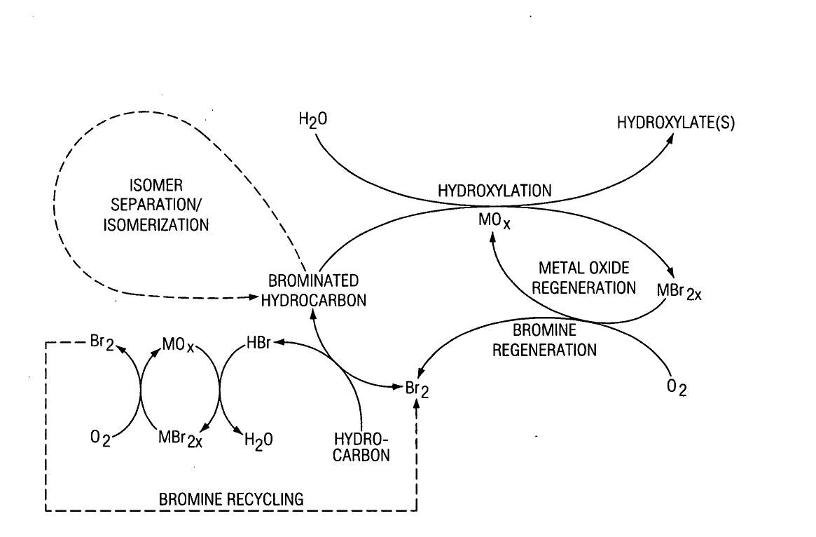

Thus, although the production of hydroxylates according to the invention can be carried out using brominated hydrocarbons purchased as commodity chemicals, it will usually be more advantageous to generate them as part of an integrated process that includes hydrocarbon bromination, metal-oxide-facilitated synthesis of a diol, triol, and/or polyol, regeneration of metal oxide, and regeneration/recycling of bromine. The process is schematically illustrated in FIG. 1.

Even natural gas can be used as a hydrocarbon feedstock in the preparation of hydroxylates and other useful compounds. In one embodiment, natural gas is brominated, producing methyl bromide and mono and di-bromides of ethane, propane and higher alkanes. This is then followed by conversion of methyl bromide into methanol, other monobromides into alcohols, and dibromides into glycols. In another embodiment, the transformation of natural gas includes bromination of natural gas, coupling to light olefins (e.g., ethylene, propylene), bromination of the olefins to dibromides, and conversion of the dibromides to glycols. In theory, carbon-carbon coupling during the bromination of natural gas or the hydroxylation step can also lead to hydroxylates.

Hydrocarbon bromination can be accomplished in a number of ways, for example, using a fixed bed reactor. The reactor may be empty or, more typically, charged with an isomerization catalyst to help generate the desired brominated isomer (see below). In an alternate embodiment, a fluidized bed or other suitable reactor is employed. A fluidized bed, for example, offers the advantage of improved heat transfer.

In one embodiment, molecular bromine (Br₂) is used to brominate a hydrocarbon in the gas or liquid phase. For example, benzene can be brominated at moderate temperatures (0 to 150° C., more preferably 20 to 75° C.) and pressures (0.1 to 200 atm, more preferably 5 to 20 atm), over the course of 1 minute to 10 hours (more preferably 15 min. to 20 hrs), using FeBr₃ or another suitable catalyst. Benzene can also be brominated using FeBr₃, in the absence of Br₂, generating mono-bromobenzene, higher bromobenzenes, hydrogen bromide, and FeBr₂. Benzene bromination is described, for example, in Organikum: Take 0.5 mol of benzene, 0.5 mol of Br₂, and 1 g of Fe-powder; add the Br₂ at room temperature (warm up to 30-40° C. if necessary to start the reaction); let stand overnight; wash with water/bisulfite, then 10% NaOH distill (vacuum). Bromobenzene yield: 65%; a residue of p-dibromobenzene is observed after distillation. (One can infer from this an ˜80% conversion of benzene, with ˜80% selectivity to the monobromide and ˜20% selectivity to the dibromide.)

In another embodiment, hydrogen bromide is used to brominate a hydrocarbon. For example, reacting an alkyne with hydrogen bromide yields a vicinal dibromide.

Brominating an aliphatic or aromatic hydrocarbon can result in a number of different compounds, having varying degrees of bromine substitution. For example, bromination of benzene can result in the formation of bromobenzene, dibromobenzene, tribromobenzene, and more highly brominated benzene compounds. However, because the boiling points of benzene (80° C.), bromobenzene (155° C.), dibromobenzene (˜220° C.), and higher brominated isomers differ significantly, the desired isomer(s) can be readily separated from benzene and other brominated isomers via distillation. The same is generally true for other bromocarbons.

Free-radical halogenation of hydrocarbons, particularly alkanes, can be non-selective in the distribution of isomers produced. With chlorine, for example, the second chlorine is likely to attack a carbon that is non-adjacent to the first chlorinated carbon atom. (e.g., 1-chlorohexane is more likely to be chlorinated at the 3 position than at the 2 position). Although this “steering” effect is less pronounced with bromine, nevertheless, free radical bromination may give the desired isomer in some cases.

More importantly, undesired isomers can often be rearranged to more desired isomers using an isomerization catalyst, such as a metal bromide (e.g., NaBr, KBr, CuBr, NiBr₂, MgBr₂, CaBr₂, etc.), metal oxide (e.g., SiO₂, ZrO₂, Al₂O₃, etc.), or metal (Pt, Pd, Ru, Ir, Rh, and the like). In addition, various isomers often have different boiling points (up to 10-15° C. difference) and can be separated using distillation.

In some cases, the desired bromide isomer is actually the thermodynamically favored product. Thus, isomerization allows one to move from the undesirable kinetic distribution of free radical bromination to a desirable thermodynamic distribution. Isomerization is especially helpful in ethane dibromination. Free radical bromination can produce as much as 75% of 1,1-dibromoethane (here the percentage refers to the distribution of the dibromide isomers). After isomerizing 1,1-dibromoethane, the thermodynamic distribution (under typical conditions of 300° C., 1 atm or higher) can be as high as 99% 1,2-dibromoethane.

Since isomerization and bromination conditions are similar, the bromination and isomerization may be accomplished in the same reactor vessel. The bromination section may be empty (no catalyst) and the isomerization section may contain the catalyst. Any monobromides that are produced can be recycled for additional bromination and isomerization. For production of a vicinal dibromide, if HBr is eliminated from the monobromide (this can occur, for example, at high temperature and low pressure, in the presence of a catalyst such as the isomerization catalysts listed above), Br₂ can be added across the double bond. Similarly, tribromides may be hydrogenated to di- or monobromides or alkane. Tribromides may also be reacted with alkane to produce mono- and dibromides (a process referred to as “reproportionation.”)

Once the desired brominated hydrocarbon(s) is obtained, the desired hydroxylate(s) is produced by allowing the brominated hydrocarbon(s) to react with water in the presence of a metal oxide, as discussed above. For example, the reaction of dibromobenzene with water in the presence of a metal oxide yields dihydroxybenzene, metal bromide, water, and possibly hydrogen bromide.

Gas Phase Production of Hydroxylates

According to one embodiment of the invention, a hydroxylated hydrocarbon having two or more hydroxyl groups is produced in the gas phase at moderate temperatures (0 to 500° C., more preferably 100 to 300° C.) and pressures (0.1 to 200 atm, more preferably 1 to 20 atm), in a fixed bed, fluidized bed, or other suitable reactor. Target reaction times are 0.1 seconds to 10 minutes, more preferably 1 to 30 seconds. Preferred and most preferred reaction parameters (temperature, pressure, time in reactor, etc.) can be selected based on reactant and product boiling points, mole fractions, reactor volume, choice of metal oxide(s), and other considerations that will become apparent to a skilled person, when considered in light of the present disclosure.

In one embodiment, water and bromocarbon(s) are introduced into a single, fixed bed, gas phase reactor charged with spherical or cylindrical metal oxide pellets. Alternatively, multiple reactors are employed, so that, as one is being regenerated, another is producing hydroxylates. Preferably, the metal oxide pellets have, on average, a longest dimension of 0.01 to 100 mm (more preferably 0.25-10 mm). Alternatively, the reactor is charged with comparably dimensioned spherical or cylindrical pellets of a suitable support material, such as zirconia, silica, titania, etc., onto which is supported the desired metal oxide(s) in a total amount of 1 to 50 wt. % (more preferably, 10 to 33 wt. %).

In another embodiment of the invention, products are generated in the gas phase in a fluidized bed reactor that contains metal oxide particles having, on average, a grain size of 5 to 5000 microns (more preferably 20 to 1500 microns).

For a gas phase reaction, hydroxylates are conveniently separated from metal bromide generated in the reactor by simply exhausting them from the reactor, leaving solid metal bromide behind. Optionally, saturated steam is introduced into the reactor to remove residual metal bromide (a process referred to as “steam stripping”), preferably at temperatures and pressures comparable to those used in the gas phase production of hydroxylates.

To regenerate the metal oxide in a fixed bed reactor, the bed is heated or cooled to a temperature of approximately 200 to 500° C., and air or oxygen (optionally preheated) at a pressure of 0.1 to 100 atm (more preferably, 0.5 to 10 atm) is introduced into the reactor. Bromine, and possibly nitrogen or unreacted oxygen, will then leave the bed. The bromine can be separated by condensation and/or adsorption and recycled for further use.

To regenerate the metal oxide in a fluidized bed reactor, solid metal oxide/metal bromide particles are removed from hydroxylates and any remaining reactants in a first cyclone. The particles are then fed into a second fluidized bed, heated or cooled to a temperature of approximately 200 to 500° C., and mixed with air or oxygen (optionally preheated) at a pressure of 0.1 to 100 atm (more preferably, 0.5 to 10 atm). Solid materials (regenerated metal oxide) are then separated from bromine, and possibly unreacted oxygen, in a second cyclone. The metal oxide particles can then be reintroduced into the first (or another) fluidized bed reactor. The bromine can be separated by condensation and/or adsorption and recycled for further use.

Liquid Phase Production of Hydroxylates

According to another aspect of the invention, a hydroxylated hydrocarbon having two or more hydroxyl groups is produced in the liquid phase at moderate temperature (0 to 500° C., preferably 100 to 300° C.) and pressure (1 to 50 atm, preferably 0.1 to 200 atm), in a semi-batch, fluidized bed, or other suitable reactor. Target reaction times are 1 minute to 24 hours (more preferably 15 minutes to 3 hours).

In one embodiment, a simple, semi-batch reactor vessel is charged with reactants and fine metal oxide particles; hydroxylates are formed; and the products are removed. Products are separated either by increasing the reactor temperature, decreasing the reactor pressure, and/or via a solvent wash. The residual solid is regenerated in the vessel.

For a liquid phase reaction carried out in a semi-batch reactor, it is preferred to use fine metal oxide particles having, on average, a grain size of 0.01-100 mm (more preferably, 0.25 to 10 mm).

In an alternate embodiment, hydroxylates are produced in the liquid phase in a fluidized bed, with liquid reactants, etc., flowing through a bed of fine metal oxide particles. The grain size of such particles is preferably 5 to 5000 microns (more preferably 20 to 1500 microns).

For a liquid phase reaction, hydroxylates are conveniently separated from metal bromide generated in the reactor using any suitable separation technique. According to one approach, hydroxylates are vaporized (and then exhausted from the reactor) by heating the metal oxide/metal bromide/reactant/product slurry, leaving solid metal bromide behind. The metal bromide is then rinsed with a suitable organic solvent, such as ethanol, removing any residual hydroxylates. In one embodiment, this is carried out at 100 to 200° C., and 5 to 200 atm.

In another embodiment, hydroxylates having sufficiently low water-solubility are separated from metal bromide by exposure to water. The metal bromide dissolves, and the water-immiscible hydroxylates are separated from the aqueous metal bromide solution (e.g., gravimetrically). The bromide solution is dried, and the solid metal bromide is then regenerated. In spray drying, the metal bromide solution is sprayed into a hot zone, forming metal bromide and steam. The metal bromide particles may be separated from the steam in a cyclone prior to being regenerated with air or oxygen.

After removal of all liquids from the reactor, the metal oxide can be regenerated in a manner essentially the same as that described above for a fixed bed, gas phase reactor.

The following examples are provided as nonlimiting embodiments of the invention. To monitor reactant conversion and product distribution, dodecane (10 wt %) is used as an internal standard for organic phases, and isopropanol is used as an internal standard for aqueous phases. Quantitation is performed using proton NMR.

### EXAMPLE 1

### Ethane to 1,2-Dibromoethane to Ethylene Glycol

A stream of ethane with a flow rate of 5 standard ml/min is passed through a bromine bubbler held at 21° C. The resulting mixture of ethane and bromine having a molar ratio of 3.6:1 (ethane:bromine) is passed through a glass reactor having a 3.6 mm inside diameter. The glass reactor is packed with borosilicate beads and the average residence time in the reactor is 27 s. 100% of the bromine is converted. 24% of the ethane is converted with selectivities of 86% to ethyl bromide, 12% to 1,2-dibromoethane, 1% to 1,1-dibromoethane, and <1% of 1,1,2-tribromoethane.

2.834 g of 1,2-dibromoethane (DBE) is reacted with 1.2 g of a fine CuO powder (Fisher Scientific, ACS grade) and 6 g of water at 150° C. in a stirred batch reactor (600 rpm) with pressure of 100 psig for 1 hour. 38% of the 1,2-DBE is converted with the following distribution:

| Product | Selectivity |
|---|---|
| Ethylene glycol (mono) | 86% |
| 2-bromoethanol | 7% |
| Ethylene | 2% |
| Acetaldehyde | 1% |
| Diethylene glycol | <2% |
| Carbon dioxide | <1% |
| Vinyl bromide | <1% |
| 1,4-dioxane | <1% |
| Unidentified | balance |

### EXAMPLE 2

### Propylene to 1,2-Dibromopropane to propylene glycol

A mixture of propane and bromine having a molar ratio of 3.6:1 (propane:bromine) is passed through a glass reactor having a 3.6 mm inside diameter. The glass reactor is packed with borosilicate beads and the average residence time in the reactor is 27 s. 100% of the bromine is converted. 28% of the propylene is converted with selectivity to 1,2-dibromopropane of greater than 99%.

1.49 g of 1,2-dibromopropane (DBP) is reacted with 0.564 g of metal oxide and 9 g of water at 150° C. in a stirred batch reactor (600 rpm) at 100 psig for 1 hr. 30% of the 1,2-DBP is converted with the following distribution:

| Product | Selectivity |
|---|---|
| 1,2-propylene glycol | 52% |
| Propylene | 17% |
| Propanal | 18% |
| Acetone | 7% |
| Propylene oxide | 2% |
| Carbon dioxide | <1% |
| 2-bromopropylene | <1% |
| 1-bromopropylene | <1% |
| Unidentified | balance |

### EXAMPLE 3

### Conversion of 1,3-Dibromopropane to 1,3-Propanediol

1.494 g of 1,3-dibromopropane is reacted with 0.564 g of metal oxide and 9 g of water at 150° C. in a stirred batch reactor (600 rpm) at 100 psig for 1 hr. 28% of the 1,3-dibromopropane is converted with the following distribution:

| Product | Selectivity |
|---|---|
| 1,3-propanediol | 83% |
| 3-bromopropanol | 10% |
| 1,6-dioxane | <2% |
| 3-bromopropylene | <2% |
| Carbon dioxide | <1% |
| Unidentified | balance |

### EXAMPLE 4

### Acetylene+HBr (Prophetic)

Acetylene and hydrogen bromide in a molar ratio of 1:5 acetylene to hydrogen bromide are passed through a reactor containing a cupric bromide catalyst, at 200° C. The reactor pressure is 5 atm and the flow rates are selected to provide a residence time of 10 s. Acetylene conversion is greater than 50% with greater than 80% selectivity to 1,2-dibromoethane; the balance is largely vinyl bromide. The 1,2-dibromoethane is separated from the products; unreacted acetylene and hydrogen bromide are recycled to the reactor; and byproduct vinyl bromide may be recycled to the reactor or hydrobrominated to 1,2-dibromoethane under approximately the same conditions with conversion of greater than 50% and selectivity approaching 100%.

### EXAMPLE 5

### Preparation of 1,2-dibromoethane Using a Doped Metal Oxide

A doped metal oxide was prepared by mixing 4 parts (by volume) of a 0.5 M solution of cupric nitrate, 1 part of a 0.5 M solution of potassium nitrate, and 5 parts of a 0.5 M solution of zirconium propoxide. The solution was stirred for more than 15 minutes, dried at 120° C., and calcined overnight (>12 h) at 500° C. The resulting compound can be represented by the formula Cu₄KZr₅Oₓ, where x is believed to be 14.5. Without being bound by theory, it is also believed that the doped metal oxide becomes Cu₄Zr₅O₁₄.KBr during the first time it is used in the hydroxylation reaction (and after the first regeneration), and the potassium remains as a bromide during subsequent cycles. In subsequent cycles, the following is believed to take place:

Reaction: Cu₄Zr₅O₁₄.KBr→CuBr₄Zr₅O₁₀KBr

Regeneration: CuBr₄Zr₅O₁₀KBr→Cu₄Zr₅O₁₄.KBr.

2.834 g of 1,2-dibromoethane (1,2-DBE) is reacted with 1 g of Cu₄KZr₅Oₓ and 6 g of water at 150° C. in a stirred batch reactor (600 rpm) at 100 psig for 1 hour. 23% of the 1,2-DBE is converted, with the following distribution:

| Product | Selectivity |
|---|---|
| Ethylene glycol (mono) | 77% |
| 2-Bromoethanol | 14% |
| Ethylene | <1% |
| Acetaldehyde | 6% |
| Diethylene glycol | <1% |
| Carbon dioxide | <1% |
| Vinyl bromide | <1% |
| 1,4-dioxane | <1% |
| Unidentified | balance |

The present invention offers the advantages of use of lower cost starting materials (e.g., alkanes and other hydrocarbons), avoidance of inefficient partial oxidation of olefins and inefficient hydration of olefin oxides, avoidance of costly water separation steps, and high to very high selectivities.

The invention has been described and illustrated by various preferred and exemplary embodiments, but is not limited thereto. Other modifications and variations will likely be apparent to the skilled person, upon reading this disclosure. The invention is limited only by the appended claims and their equivalents.

What is claimed is:

1. A method of making a hydroxylated hydrocarbon having two or more hydroxyl groups, comprising:
allowing a brominated hydrocarbon having at least two bromine atoms to react with water or steam in the presence of a metal oxide to form a hydroxylated hydrocarbon having two or more hydroxyl groups.

2. A method as recited in claim 1, wherein the brominated hydrocarbon is saturated.

3. A method as recited in claim 1, wherein the brominated hydrocarbon is unsaturated.

4. A method as recited in claim 1, wherein the brominated hydrocarbon is aliphatic.

5. A method as recited in claim 4, wherein the brominated hydrocarbon comprises an alkyl dibromide.

6. A method as recited in claim 5, wherein the alkyl dibromide is selected from the group consisting of 1,2-dibromoethane, 1,2-dibromopropane, 1,3-dibromopropane, 1,4-dibromobutane, and mixtures thereof.

7. A method as recited in claim 4, wherein the brominated hydrocarbon comprises 1,4-dibromo-2-butene.

8. A method as recited in claim 4, wherein the brominated hydrocarbon comprises 1,2,3-tribromopropane.

9. A method as recited in claim 4, wherein the brominated hydrocarbon comprises 2,3,4,5-tetrabromohexane.

10. A method as recited in claim 4, wherein the brominated hydrocarbon comprises a poly-brominated polyolefin having a molecular weight prior to bromination of about 250-5000.

11. A method as recited in claim 1, wherein the brominated hydrocarbon is aromatic.

12. A method as recited in claim 11, wherein the brominated hydrocarbon is selected from the group consisting of o-, m-, and p-dibromobenzene, and mixtures thereof.

13. A method as recited in claim 1, wherein the brominated hydrocarbon is mixed aliphatic-aromatic.

14. A method as recited in claim 1, wherein the brominated hydrocarbon has the formula
BrCH₂CH(Br)—R, where R is hydrogen, C₁₋₁₆ alkyl, or phenyl.

15. A method as recited in claim 1, wherein the brominated hydrocarbon has the formula
R—CH(Br)—CH(Br)—R′, where R and R′ are independently selected from the group consisting of hydrogen, C₁₋₁₆ alkyl, and phenyl.

16. A method as recited in claim 1, wherein the metal oxide is selected from the group consisting of copper oxide, magnesium oxide, yttrium oxide, nickel oxide, cobalt oxide, iron oxide, calcium oxide, vanadium oxide, molybdenum oxide, chromium oxide, manganese oxide, zinc oxide, lanthanum oxide, tungsten oxide, tin oxide, indium oxide, bismuth oxide, and mixtures thereof.

17. A method as recited in claim 16, wherein the metal oxide is doped with one or more alkali metals.

18. A method as recited in claim 1, wherein the metal oxide is alkali-doped.

19. A method as recited in claim 1, wherein the metal oxide is selected from the group consisting of CuO, MgO, Y₂O₃, NiO, Co₂O₃, Fe₂O₃, and mixtures thereof.

20. A method as recited in claim 1, wherein the metal oxide comprises one or more alkali metal-doped mixed copper, magnesium, yttrium, nickel, cobalt, or iron oxide.

21. A method as recited in claim 20, wherein the metal oxide(s) is doped to contain 5-20 mol % alkali.

22. A method as recited in claim 1, wherein the metal oxide is doped with one or more alkali metal bromides.

23. A method as recited in claim 22, wherein the metal oxide is doped to contain 5-20 mol % alkali.

24. A method as recited in claim 1, wherein the metal oxide is supported on zirconia, titania, alumina, silica, or another suitable support material.

25. A method as recited in claim 1, further comprising regenerating the metal oxide using air or oxygen.

26. A method as recited in claim 1, further comprising separating the hydroxylated hydrocarbon from any metal bromide formed in the reaction of the brominated hydrocarbon water or steam.

27. A method of making a hydroxylated hydrocarbon having two or more hydroxyl groups, comprising:
(a) brominating a hydrocarbon to form a brominated hydrocarbon having at least two bromine atoms; and
(b) forming a hydroxylated hydrocarbon having two or more hydroxyl groups by allowing the brominated hydrocarbon to react with water or steam in the presence of a metal oxide.

28. A method as recited in claim 27, further comprising separating the hydroxylated hydrocarbon from any metal bromide formed in the reaction of the brominated hydrocarbon water or steam.

29. A method as recited in claim 28, further comprising regenerating the metal oxide and bromine by allowing the metal bromide to react with air or oxygen.

30. A method as recited in claim 27, wherein the hydrocarbon is brominated using Br₂.

31. A method as recited in claim 27, wherein the hydrocarbon comprises natural gas.

32. A method as recited in claim 27, wherein the hydrocarbon comprises an alkane.

33. A method as recited in claim 27, wherein the hydrocarbon comprises an alkene.

34. A method as recited in claim 27, wherein the hydrocarbon comprises an alkyne.

35. A method as recited in claim 34, wherein the hydrocarbon is brominated using hydrogen bromide.

36. A method of making a hydroxylated hydrocarbon having two or more hydroxyl groups, comprising:
(a) forming hydrogen bromide by allowing molecular bromine to react with molecular hydrogen or a source of hydrogen atoms;
(b) forming a brominated hydrocarbon having at least two bromine atoms by allowing an alkyne to react with the hydrogen bromide; and
(c) forming a hydroxylated hydrocarbon having two or more hydroxyl groups by allowing the brominated hydrocarbon to react with water or steam in the presence of a metal oxide.

37. A method as recited in claim 36, further comprising regenerating the metal oxide and/or hydrogen bromide.

38. A method of making a hydroxylated hydrocarbon having a formula R—CH(OH)CH(OH)—R′, where R and R′ are independently selected from the group consisting of hydrogen, C₁₋₁₆ alkyl, and phenyl, comprising:
allowing a brominated hydrocarbon having a formula R—CH(Br)—CH(Br)—R′, where R and R′ are independently selected from the group consisting of hydrogen, C₁₋₁₆ alkyl, and phenyl, to react with water or steam in the presence of a metal oxide to form the hydroxylated hydrocarbon.
